(19) United States
(12) Patent Application Publication
(10) Pub. No.: US 2010/0173837 A1
(43) Pub. Date: Jul. 8, 2010

(54) DOSING FORMS AND REGIMENS COMPRISING 3-[(R)-2-(N,N-DIMETHYLAMINO)ETHYLTHIO-SAR]-4-(GAMMAHYDROXYMETHYLLEUCINE)CYCLOSPORINE

(75) Inventor: Samuel Earl Hopkins, Research Triangle Park, NC (US)

Correspondence Address: BUCHANAN, INGERSOLL & ROONEY PC, POST OFFICE BOX 1404, ALEXANDRIA, VA, 22313-1404, US

(73) Assignee: Scynexis, Inc., Research Triangle Park, NC (US)

(21) Appl. No.: 12/683,662

(22) Filed: Jan. 7, 2010

Related U.S. Application Data
(60) Provisional application No. 61/143,062, filed on Jan. 7, 2009.
(60) Provisional application No. 61/156,026, filed on Feb. 27, 2009.

(51) Int. Cl. A61K 38/13; A61P 31/12

(52) U.S. Cl. 514/11

(57) ABSTRACT

Provided herein are specific doses of, and dosing regimens for, using SCY-635 in treating or preventing diseases, in particular hepatitis C virus (HCV) infection.

### CROSS REFERENCE TO RELATED APPLICATIONS

The present application claims the benefit of priority of U.S. Provisional Application No. 61/143,062, filed Jan. 7, 2009 and U.S. Provisional Application No. 61/156,026, filed Feb. 27, 2009, the contents of which are hereby incorporated by reference in their entireties and relied upon.

### FIELD OF THE INVENTION

The present invention provides specific doses of, and dosing regimens for, SCY-635 in treating or preventing diseases, in particular hepatitis C virus (HCV) infections.

### BACKGROUND OF THE INVENTION

3-Substituted ether and thioether cyclosporines and their use to treat, prevent or manage certain viral infections are known in the literature. One such compound is SCY-635, which is 3-[(R)-2-(N,N-dimethylamino)ethylthio-Sar]-4-(gammahydroxymethylleucine)cyclosporine. The use of SCY-635 to treat HIV or AIDS is described in U.S. Pat. No. 5,994,299, and its use to treat HCV is described in U.S. Pat. No. 7,196,161. SCY-635 exhibits potent and selective inhibition of HCV-specific RNA replication in both the subgenomic and full length replicon systems in the absence of immunosuppressive activity (Li, K., et al. (2006) “Preclinical evaluation of SCY-635, a cyclophilin inhibitor with potent anti-HCV activity,” Abstract number 934, American Association for the Study of Liver Disease). As with all drugs, proper doses and dosing regimens for treating human subjects having diseases including HIV and HCV are essential for achieving a desired or optimal therapeutic effect without adverse or unwanted effects.

Therefore, a need exists for safe, effective, and non-toxic doses and dosing regimens that either prevent or reduce any adverse or unwanted effects or provide an optimal therapeutic effect or both, that is, provide a desirable therapeutic profile.

### SUMMARY OF THE INVENTION

Provided herein are dosing regimens wherein specific doses of SCY-635, or a pharmaceutically acceptable salt, solvate or hydrate thereof, are administered at specific time intervals to treat diseases, in particular viral diseases such as HIV, and in particular HCV. Also provided herein are specific doses and unit dosage forms of SCY-635, or a pharmaceutically acceptable salt, solvate or hydrate thereof.

In one embodiment, provided herein are methods for treating, preventing or managing hepatitis C virus infection in a human subject infected with, or at risk for infection with, hepatitis C virus, the method comprising administering to the human subject an effective amount of SCY-635, or a pharmaceutically acceptable salt, solvate or hydrate thereof, at least two times in the course of a 24 hour period. Also provided herein are methods for administering to an infected human subject in need thereof a pharmaceutical composition comprising an effective amount of SCY-635, or a pharmaceutically acceptable salt, solvate or hydrate thereof, two or three times in the course of a 24 hour period.

### BRIEF DESCRIPTION OF THE FIGURES

FIG. 1 depicts the median plasma concentration-time profile following administration of a single dose of SCY-635 (Compound 1) to HCV-infected human subjects on Day 1 at different dose levels, as described in Examples 4 and 5 below.

FIG. 2 depicts the median plasma concentration-time profile following administration of SCY-635 (Compound 1) to HCV-infected human subjects three times a day (t.i.d) at different dose levels, as described in Examples 4 and 5 below.

FIG. 3 depicts the mean and median log₁₀ baseline adjusted plasma HCV RNA profiles for SCY-635 administered at 300 mg t.i.d (n=6) plotted together with the mean and median profiles for placebo human subjects (n=3), as described in Examples 4 and 6 below.

FIG. 4 depicts the mean plasma concentration-time profiles following administration of a single dose of SCY-635 (Compound 1) to non-infected human subjects on Day 1 at dose levels of 400, 500 and 600 mg, as described in Example 7 below.

FIG. 5 depicts the mean plasma concentration-time profile following administration of SCY-635 (Compound 1) given as 500 mg b.i.d for 14 consecutive days to non-infected human subjects, as described in Example 8 below.

### DETAILED DESCRIPTION

Provided herein are doses and dosage regimens for delivering SCY-635. Cyclosporines are cyclophilin-inhibitors and are believed to be indirect inhibitors of the viral NS5B polymerase enzyme. It is believed that cyclosporines are capable of preventing association of NS5B polymerase with host cyclophilins, such as cyclophilin A and cyclophilin B; see for example Watashi et al., Reviews in Medical Virology, (DOI: 10.1002/rmv) February 2007. In this specification it will be understood that cyclosporine derivatives are not necessarily direct NS5B polymerase inhibitors. Cyclophilin A is believed to be capable of binding to a region of the HIV-1-capsid protein centered around the glycine 89-proline 90 peptide bond within the HIV-1 capsid (p24). In steps that follow viral entry, the capsid core is believed to undergo an ordered uncoating to deliver the viral genome into the cytosol of susceptible cells. Uncoating is believed to require recruitment of host cyclophilin A. The results of in vitro studies indicate that SCY-635 is capable of binding to cyclophilin A and inhibiting its intrinsic peptidyl prolyl isomerase activity (Li et al., (2006), “SCY-635 and Related Cyclophilin inhibitors as Antiretroviral Agents,” Abstract number H-0255, Interscience Conference on Antimicrobial Agents and Chemotherapy). Taken together these results suggest that SCY-635 is capable of inhibiting HIV-1 infectivity by binding to host cyclophilin A, thereby preventing uncoating of the viral core.

The pharmacokinetics of cyclosporine A and certain derivatives are discussed in the literature, see for example Foxwell et al, Biophysica acta, Volume 938(3), pages 447-455 (1988); and Awni W. and Sauchuk R, Drug Metabolism & Disposition, Volume 13(2), pages 133-135 (1985). A particular problem in the dosing of cyclosporine derivatives can be maintaining a plasma concentration of cyclosporine derivative in a human subject due to partitioning of the compound between whole blood cells and plasma. It has been found that unless a substantially saturated whole blood cell compartment can be maintained throughout the delivery of the cyclosporine derivative, it might not be possible to maintain therapeutically relevant concentrations of the cyclosporine derivative in the plasma. According to the methods provided herein, certain divided dosing regimens can permit the maintenance of plasma concentrations of SCY-635 sufficient to confer anti-viral activity, while maintaining a satisfactory safety profile.

In one embodiment, provided herein are methods for treating, preventing or managing HCV infection in a human subject infected with, or at risk for infection with, HCV, the method comprising administering to the human subject an effective amount of SCY-635, or a pharmaceutically acceptable salt, solvate or hydrate thereof, as a divided dose in the course of a 24 hour period. In a further embodiment, provided herein are methods for treating, preventing or managing HIV infection in a human subject infected with, or at risk for infection with, HIV, the method comprising administering to the human subject an effective amount of SCY-635, or a pharmaceutically acceptable salt, solvate or hydrate thereof, two or three times in the course of a 24 hour period. In a still further embodiment the human subject is co-infected with HCV and HIV. Preferably, the administration is made two or three times per day continually, for a number of days, weeks or months. Infection or risk for infection can be determined according to any technique deemed suitable by the practitioner of skill in the art.

It will be understood that as used herein, reference to amounts of SCY-635 refer to the amount of free base (i.e. 3-[(R)-2-(N,N-dimethylamino)ethylthio-Sar]-4-(gammahydroxymethylleucine)cyclosporine).

In a further embodiment, provided herein are methods for treating, preventing or managing HCV infection in a human subject infected with, or at risk for infection with, HCV, the method comprising administering to the human subject a pharmaceutical composition comprising an effective amount of SCY-635, or a pharmaceutically acceptable salt, solvate or hydrate thereof, at least two times in the course of a 24 hour period. In a still further embodiment, the administration is made two or three times per day continually, for a number of days, weeks or months.

In a further embodiment, provided herein are methods of administering SCY-635, or a pharmaceutically acceptable salt, solvate or hydrate thereof, wherein the active agent is administered to an infected human subject in need thereof at least two times in a 24 hour period, wherein each administration is preferably separated by about 4 to about 14 hours.

In another embodiment, provided herein are methods for continual therapy wherein SCY-635, or a pharmaceutically acceptable salt, solvate or hydrate thereof, is administered to an infected human subject in need thereof for a certain period of time (e.g., 5, 7, 10, 14, 20, 24, 28, 60, 120, 360 days or longer).

In another embodiment, provided herein are methods for the administration of SCY-635, or a pharmaceutically acceptable salt, solvate or hydrate thereof, in divided doses (e.g., two or three times daily) of between about 4 mg/kg and about 50 mg/kg; between about 10 mg/kg and about 50 mg/kg; between about 10 mg/kg and about 34 mg/kg; between about 13 mg/kg and about 27 mg/kg; between about 14 mg/kg and about 20 mg/kg; between about 15 mg/kg and about 19 mg/kg; or between about 15 mg/kg and about 18 mg/kg, to a human subject infected with, or at risk for infection with, HCV. In a particular embodiment, SCY-635, or a pharmaceutically acceptable salt, solvate or hydrate thereof, is administered in a dose of about 10 mg/kg, about 13 mg/kg, about 14 mg/kg, about 15 mg/kg, about 17 mg/kg or about 18 mg/kg. In another embodiment, any dose of the SCY-635, or a pharmaceutically acceptable salt, solvate or hydrate, described in the preceding embodiment is administered two or three times in a 24 hour period.

In another embodiment, provided herein are methods for treating a human subject infected with HCV, which include administering to the human subject SCY-635 (a) in an amount of about 200 mg each time, 3 times per day; (b) in an amount of about 250 mg each administration, 3 times per day; (c) in an amount of about 280 mg each administration, 3 times per day; (d) in an amount of about 300 mg each time, 3 times per day; (e) in an amount of about 330 mg each time, 3 times per day; (f) in an amount of about 350 mg each time, 3 times per day; (g) in an amount of about 400 mg each time, 3 times per day; (h) in an amount of about 500 mg each time, 3 times per day; or (i) in an amount of about 600 mg each time, 3 times per days. In one aspect of the above embodiments, SCY-635 is administered to a human subject once every 8 hours. In another aspect of the above embodiments, SCY-635 is administered at 7-, 7- and 10-hour intervals per day (e.g. at about 7:00 AM, about 2:00 PM, and at about 9:00 PM).

In another embodiment, provided herein are methods for treating a human subject infected with HCV, which include administering to the human subject SCY-635 (a) in an amount of about 300 mg each time, 2 times per day, once every 12 hours; (b) in an amount of about 400 mg each administration, 2 times per day, once every 12 hours; (c) in an amount of about 425 mg each administration, 2 times per day, once every 12 hours; (d) in an amount of about 450 mg each time, 2 times per day, once every 12 hours; (e) in an amount of about 500 mg each time, 2 times per day, once every 12 hours; (f) in an amount of about 550 mg each time, 2 times per day, once every 12 hours; (g) in an amount of about 600 mg each time, 2 times per day, once every 12 hours; (h) in an amount of about 625 mg each time, 2 times per day, once every 12 hours; (i) in an amount of about 650 mg each time, 2 times per day, once every 12 hours; (j) in an amount of about 700 mg each time, 2 times per day, once every 12 hours; or (k) in an amount of about 800 mg each time, 2 times per day, once every 12 hours.

A method that provides greater than about 600 mg of SCY-635, given as a divided dose over a 24 hour period, can effectively result in a high trough level of SCY-635 in plasma. As used herein, “trough level” refers to the lowest level that a medicine is present in the body. It can be important, particularly in viral diseases, to maintain drug levels above a certain concentration to maintain appropriate inhibition of viral replication. In particular, it has been found that the a dosing regimen of greater than about 200 mg of SCY-635 each time, three times a day, once every 8 hours, can lead to disproportionately higher trough levels of SCY-635 than seen at lower daily doses.

In another embodiment, provided herein are methods for treating a human subject infected with HCV, given as a divided dose over a 24 hour period, which include administering to the human subject SCY-635 (a) in an amount of from 800 to 999 mg per day; (b) in an amount of from 810 to 997 mg per day; (c) in an amount of from 820 to 995 mg per day; (d) in an amount of from 850 to 950 mg per day; (e) in an amount of 870 to 930 mg per day; (f) in an amount of from 880 to 920 mg per day; or (g) in an amount of from 890 to 910 mg per day. In one aspect of these embodiments SCY-635 is given in two doses over a 24 hour period. In another aspect of these embodiments SCY-635 is given in three doses over a 24 hour period.

In another embodiment, provided herein are methods for treating a human subject infected with HCV, given as a divided dose over a 24 hour period, which include administering to the human subject SCY-635 (a) in an amount of from about 600 to about 1050 mg per day; (b) in an amount of from about 600 to about 1000 mg per day; (c) in an amount of from about 750 to about 1000 mg per day; (d) in an amount of from about 800 to about 1000 mg per day; or (e) in an amount of from about 900 to about 1000 mg per day. In one aspect of these embodiments SCY-635 is given in two doses over a 24 hour period. In another aspect of these embodiments SCY-635 is given in three doses over a 24 hour period.

In another embodiment, provided herein are methods for treating a human subject infected with HCV, which include administering to the human subject SCY-635, given as a divided dose over a 24 hour period, (a) in an amount of at least 1001 mg per day; (b) in an amount of at least 1003 mg per day; (c) in an amount of at least 1005 mg per day; (d) in an amount of from 1010 to 1200 mg per day; (e) in an amount of from 1020 to 1200 mg per day; (e) in an amount of 1040 to 1150 mg per day; (f) in an amount of from 1050 to 1120 mg per day; or (g) in an amount of from 1060 to 1100 mg per day. In one aspect of these embodiments SCY-635 is given in two doses over a 24 hour period. In another aspect of these embodiments SCY-635 is given in three doses over a 24 hour period.

In one embodiment SCY-635 is given in two doses over a 24 hour period and the time between doses is from about 8 hours to about 16 hours. In another embodiment SCY-635 is given in two doses over a 24 hour period and the time between doses ranges from about 10 hours to about 14 hours.

In one embodiment SCY-635 is given in three doses over a 24 hour period and the time between doses is from about 4 hours to about 12 hours. In another embodiment SCY-635 is given in three doses over a 24 hour period and the time between doses ranges from about 6 hours to about 10 hours.

In another embodiment, a therapeutically effective plasma concentration of SCY-635 is obtained and a certain trough level concentration of SCY-635 is maintained at steady state. These methods can be particularly useful for treating a human infected with HCV by administering a SCY-635 formulation, wherein a trough SCY-635 plasma level is maintained at a minimum of about 110 ng/mL, about 115 ng/mL, about 135 ng/mL, about 216 ng/mL, or about 400 ng/mL, over a 24 hour period at steady state. In certain embodiments, the methods can be particularly useful for treating a human suffering from HCV infection by administering a SCY-635 formulation, wherein the trough SCY-635 plasma level is maintained at a minimum of about 115 ng/mL over a 24 hour period at steady state. In certain embodiments, the methods can be particularly useful for treating, preventing or managing HCV infection in a human subject infected with, or at risk for infection with, HCV, wherein the compound is administered in amount sufficient to maintain a trough plasma concentration of the compound of greater than about 115 ng/ml at steady state.

A relatively rapid increase in plasma concentration can be obtained by administering a loading dose to a human subject. In one embodiment, the loading dose is about 400 mg of SCY-635. In another embodiment the loading dose is about 600 mg of SCY-635. In a further embodiment the loading dose is about 800 mg of SCY-635. In another embodiment the loading dose is about 900 mg of SCY-635. In another embodiment the loading dose is about 1000 mg of SCY-635. In a further embodiment, a loading dose of about 400 mg of SCY-635 is administered, followed by about 300 mg of SCY-635, administered two times a day. In a further embodiment, a loading dose of about 400 mg of SCY-635 is administered, followed by about 300 mg of SCY-635, administered three times a day.

In one embodiment, provided herein is a dosage form (other than the dosage form used to administer the loading dose) comprising about 300 mg of SCY-635, and the dosage form can be administered three times a day (e.g. t.i.d). In other embodiments, the dosage form comprises about 300 mg of SCY-635 once every 8 hours (i.e. q8h).

In certain embodiments, the SCY-635 dosage form can be administered once every 8 hours. In other embodiments, the SCY-635 dosage form can be administered once every 7, 7 and 10 hours (e.g. at about 7:00 AM, about 2:00 PM, and at about 9:00 PM).

In certain embodiments, the treatment duration with SCY-635 is shorter than the current standard of care.

In certain embodiments, SCY-635 is administered for less than about 182 days.

In certain embodiments, SCY-635 is administered for about 91 days.

In certain embodiments, SCY-635 is administered for about 28 days.

In another embodiment, provided herein are unit dosage formulations that comprise between about 600 mg and about 2000 mg, between about 800 mg and about 1600 mg, between about 850 mg and about 1200 mg, between about 850 mg and about 1100 mg, between about 900 mg and about 1100 mg, or between about 900 mg and about 1050 mg of SCY-635, or a pharmaceutically acceptable salt, solvate or hydrate thereof. In certain embodiments, provided herein are unit dosage formulations that comprise between about 800 mg and about 1600 mg of SCY-635, or a pharmaceutically acceptable salt, solvate or hydrate thereof.

In another embodiment, provided herein are unit dosage formulations that comprise about 100 mg, about 120 mg, about 150 mg, about 175 mg, about 200 mg, about 250 mg, about 280 mg, about 300 mg, about 330 mg, about 350 mg, about 400 mg, about 500 mg, about 550 mg, about 600 mg, about 625 mg, about 650 mg, about 700 mg, about 750 mg, about 900 mg, about 1000 mg, about 1050 mg, about 1200 mg, about 1250 mg, about 1600 mg or about 2000 mg of SCY-635, or a pharmaceutically acceptable salt, solvate or hydrate thereof. In a preferred embodiment, provided herein are unit dosage formulations that comprise about 200 mg, about 300 mg, about 350 mg, about 400 mg or about 500 mg of SCY-635, or a pharmaceutically acceptable salt, solvate or hydrate thereof.

In another embodiment, provided herein are methods for maintaining a steady state average plasma concentration of SCY-635, or a pharmaceutically acceptable salt, solvate or hydrate thereof, of greater than about 250 ng/ml, about 275 ng/ml, about 300 ng/ml, about 350 ng/ml, about 475 ng/ml, or about 900 ng/ml, in a human subject for at least about 4, 6, 8, 12 or 24 hours or longer, comprising administering an effective amount of SCY-635, or a pharmaceutically acceptable salt, solvate or hydrate thereof, to a human subject infected with, or at risk for infection with, HCV. In certain embodiments, provided herein are methods for maintaining a steady state average plasma concentration of SCY-635, or a pharmaceutically acceptable salt, solvate or hydrate thereof, of greater than about 250 ng/ml in a human subject for at least about 4, 6, 8, 12 or 24 hours or longer, comprising administering an effective amount of SCY-635, or a pharmaceutically acceptable salt, solvate or hydrate thereof, to a human subject infected with, or at risk for infection with, HCV.

Without being limited by any theory, provided herein, in part, are specific doses and dosing regimens for SCY-635, or a pharmaceutically acceptable salt, solvate or hydrate thereof, for the treatment of HCV or HIV.

In certain embodiments, the HCV is genotype 1 and can be of any subtype. For instance, in certain embodiments, the HCV is subtype 1a, 1b or 1c. It is believed that HCV infection of genotype 1 responds poorly to current interferon therapy. The methods provided herein can be advantageous for therapy of HCV infection with genotype 1.

In certain embodiments, the HCV is other than genotype 1. In certain embodiments, the HCV is genotype 2 and can be of any subtype. For instance, in certain embodiments, the HCV is subtype 2a, 2b or 2c. In certain embodiments, the HCV is genotype 3 and can be of any subtype. For instance, in certain embodiments, the HCV is subtype 3a, 3b or 10a. In certain embodiments, the HCV is genotype 4 and can be of any subtype. For instance, in certain embodiments, the HCV is subtype 4a. In certain embodiments, the HCV is genotype 5 and can be of any subtype. For instance, in certain embodiments, the HCV is subtype 5a. In certain embodiments, the HCV is genotype 6 and can be of any subtype. For instance, in certain embodiments, the HCV is subtype 6a, 6b, 7b, 8b, 9a or 11a. See, e.g., Simmonds, 2004, J Gen Virol. 85:3173-88; Simmonds, 2001, J. Gen. Virol., 82, 693-712, the contents of which are incorporated by reference in their entirety.

The methods provided herein include the treatment, prevention and management of diseases while reducing or avoiding adverse or unwanted effects, e.g., toxicities or side effects. SCY-635 may be administered by any conventional route, in particular orally, parenterally, rectally or by inhalation (e.g., in the form of aerosols). The preferred route of administration for the doses and dosing regimens described herein is oral.

In one embodiment, provided herein is a method of administering SCY-635, or a pharmaceutically acceptable salt, solvate or hydrate thereof, wherein the active agent is administered to an infected human subject in need thereof two times in a 24 hour period, wherein each administration is preferably separated by about 4-6 to 14 hours; and in one embodiment each administration is preferably separated by about 12 hours. In these embodiments, the active agent can be administered, for example, at meal time, such as breakfast and supper.

In another embodiment, provided herein is a method of administering SCY-635, or a pharmaceutically acceptable salt, solvate or hydrate thereof, wherein the active agent is administered to an infected human subject in need thereof three times in a 12 or 24 hour period, wherein each administration is preferably separated by about 4 to 14 hours. In a particular embodiment, the active agent is administered once in the morning, once in the afternoon and once in the evening. Preferred intervals between doses include 4, 5, 6, 7, 8, 9, 10, 11, 12, 13 and 14 hours.

In a particularly preferred dosing regimen, a human subject is administered the active agent within 30 minutes after a meal at approximately 7-, 7-, and 10-hour intervals (e.g. at about 7:00 AM after breakfast, about 2:00 PM after lunch, and at about 9:00 PM after supper).

In yet another embodiment, provided herein are methods for the administration of SCY-635, or a pharmaceutically acceptable salt, solvate or hydrate thereof, in divided (e.g., two or three times in a 24 hour period) doses between about 10 mg/kg and about 50 mg/kg, between about 10 mg/kg and about 34 mg/kg, between about 13 mg/kg and about 27 mg/kg, between about 14 mg/kg and about 20 mg/kg, between about 14 mg/kg and about 19 mg/kg, between about 15 mg/kg and about 19 mg/kg, or between about 15 mg/kg and about 17.5 mg/kg to an infected human subject in need thereof. In a particular embodiment, SCY-635, or a pharmaceutically acceptable salt, solvate or hydrate thereof, is administered at a dose of about 14-20 mg/kg, about 14-19 mg/kg, about 15-19 mg/kg, or about 15-17 mg/kg. In a particular embodiment, SCY-635, or a pharmaceutically acceptable salt, solvate or hydrate thereof, is administered at a dose of about 10 mg/kg, about 13 mg/kg, about 14 mg/kg, about 15 mg/kg, about 18 mg/kg, about 19 mg/kg, about 20 mg/kg, about 33 mg/kg, about 34 mg/kg, or about 50 mg/kg. In another embodiment, any dose of SCY-635 or a pharmaceutically acceptable salt, solvate or hydrate described in the preceding embodiment is administered three times in a 24 hour period.

In another embodiment, provided herein are methods for continual therapy wherein SCY-635, or a pharmaceutically acceptable salt, solvate or hydrate thereof, is administered daily to an infected human subject in need thereof for a certain period of time (e.g., 5, 7, 10, 14, 20, 24, 28, 60 or 120 days or more). In one embodiment, the active agent is continually administered three times per 24 hour period. In a specific embodiment, the active agent is continually administered three times per 24 hour period at doses of about 10 mg/kg, about 13 mg/kg, about 15 mg/kg or about 18 mg/kg for days, weeks, months or years. In a specific embodiment, the active agent is continually administered three times per 24 hour period at doses of about 13 mg/kg, about 14 mg/kg, about 15 mg/kg, about 18 mg/kg, or about 19 mg/kg for days, weeks or months. In a specific embodiment, the active agent is continually administered three times per 24 hour period at doses of about 14 mg/kg, about 15 mg/kg or about 18 mg/kg for days, weeks or months.

In one embodiment, SCY-635 is administered at an amount effective to achieve at least a 0.7 log₁₀ decrease in HCV RNA in the plasma. In another embodiment, SCY-635 is administered at an amount effective to achieve at least a 0.8 log₁₀ decrease in HCV RNA in the plasma. In a further embodiment, SCY-635 is administered at an amount effective to achieve at least a 1.8 log₁₀ decrease in HCV RNA in the plasma.

In certain embodiments, SCY-635 or a pharmaceutically acceptable salt, solvate or hydrate thereof may be administered according to the doses and dosing regimens described herein in combination with a one or more additional active agents (e.g., simultaneously or sequentially). In particular embodiments, SCY-635 or a pharmaceutically acceptable salt, solvate or hydrate thereof may be administered according to the doses and dosing schedules described herein in combination with the one or more additional active agents. The administration of the additional active agent(s) may be topical, enteral (e.g. oral, duodenal, rectal), parenteral (e.g., intravenous, intraarterial, intramuscular, subcutaneous, intradermal or interaperitoneal) or intrathecal.

The additional active agents may be useful to treat HCV and include but are not limited to immunomodulatory agents, inhibitors of HCV NS3-NS4B protease, HCV polymerase, HCV protein, HCV entry, HCV assembly, HCV NS5A protein, HCV NS5B protein, inhibitors of another target in the HCV life cycle and other anti-HCV agents, including but not limited to nucleoside analogs for the treatment of HCV infection, ribavirin analogs such as rebetol, copegus and viramidine (taribavirin), amantadine, and telbivudine; or nitazoxanide.

Examples of NS3-NS4A protease inhibitors are described in WO 99/07733, WO 99/07734, WO 00/09558, WO 00/09543, WO 00/59929, WO 03/064416, WO 03/064455, WO 03/064456, WO 2004/030670, WO 2004/037855, WO 2004/039833, WO 2004/101602, WO 2004/101605, WO 2004/103996, WO 2005/028501, WO 2005/070955, WO 2006/000085, WO 2006/007700, WO 2006/007708, WO 2007/009227, WO 02/060926, WO 03/053349, WO 03/099274, WO 03/099316, WO 2004/032827, WO 2004/043339, WO 2004/094452, WO 2005/046712, WO 2005/051410, WO 2005/054430 (all by BMS), WO 2004/072243, WO 2004/093798, WO 2004/113365, WO 2005/010029, WO 2005/037214, WO 01/77113, WO 01/81325, WO 02/08187, WO02/08198, WO 02/08244, WO 02/08256, WO 02/48172, WO 03/062228, WO 03/062265, WO 2005/021584, WO 2005/030796, WO 2005/058821, WO 2005/051980, WO 2005/085197, WO 2005/085242, WO 2005/085275, WO 2005/087721, WO 2005/087725, WO 2005/087730, WO 2005/087731, WO 2005/107745 and WO 2005/113581, WO 2006/119061, WO 2007/016441, WO 2007/015855, WO 2007/015787, and WO 2006/043145, all of which are herein incorporated by reference; and the candidates telaprevir (VX-950), boceprevir, ITMN-191, MK-7009, PF-00868554, TMC435350, SCH900518, MK70009, BILN-2061, BILN-2065, or BMS-605339.

Inhibitors of HCV polymerase include agents (compounds or biologicals) that are effective to inhibit the function of an HCV polymerase. Such inhibitors include, but are not limited to, non-nucleoside and nucleoside inhibitors of NS4A, NS5A and NS5B polymerase. Examples of inhibitors of HCV polymerase include but are not limited to those compounds described in: WO 02/04425, WO 03/007945, WO 03/010140, WO 03/010141, WO 2004/064925, WO 2004/065367, WO 2005/080388, WO 2006/007693, WO 2007/019674, WO 2007/087717, WO 01/47883, WO 03/000254, WO 2007/033032, WO 2007/033175, WO 2006/020082, US 2005/0119318, WO 2005/034850, WO 03/026587, WO 2007/092000, WO 2007/143521, WO 2007/136982, WO 2007/140254, WO 2007/140200, WO 2007/092888, WO 2007/095269, WO 2007/054741, WO 03/062211, WO 99/64442, WO 00/06529, WO 2004/110442, WO 2005/034941, WO 2006/119975, WO 2006/046030, WO 2006/046039, WO 2005/023819, WO 02/06246, WO 2007/065883, WO 2007/129119, WO 2007/029029, WO 2006/029912, WO 2006/027628, WO 2007/028789, WO 2006/008556, WO 2004/087714, WO 2005/012288, WO 2005/014543, WO 2005/049622 (Japan Tobacco), and WO 2005/121 132, WO 2005/080399, WO 2006/052013, WO 2006/119646, WO 2007/039146, WO 2005/021568, WO 2006/094347 (Biota), WO 2006/093801, WO 2005/019191, WO 2004/041818, US 2004/0167123, US 2005/0107364), WO 2007/034127 (all of which are herein incorporated by reference); and the candidates MK-0608, VCH-759, VCH-916, VCH-222, PF-868554, GS9190, NM283 (valopicitabine), PSI-6130, NM-107, R7128, GSK625433, R803, R-1626, BILB-1941, JTK-109 and JTK-003.

Still other agents include, but are not limited to: PEG-INTRON® (peginterferon alpha-2b, available from Schering Corporation, Kenilworth, N.J.); INTRON-A®, (VIRAFERON®, interferon alpha-2b available from Schering Corporation, Kenilworth, N.J.); ribavirin (1-beta-D-ribofuranosyl-1H-1,2,4-triazole-3-carboxamide, available from Valeant Pharmaceuticals, Inc.); Albuferon™ (albumin-Interferon alpha) available from Human Genome Sciences REBETROL® (Schering Corporation, Kenilworth, N.J.); COPEGUS® (Hoffmann-La Roche, Nutley, N.J.); PEGASYS® (peginterferon alpha-2a available from Hoffmann-La Roche); ROFERON® (recombinant interferon alpha-2a available from Hoffmann-La Roche); BEREFOR® (interferon alpha 2 available from Boehringer Ingelheim Pharmaceutical, Inc); SUMIFERON® (a purified blend of natural alpha interferons such as Sumiferon available from Sumitomo, Japan); ALFERON® (a mixture of natural alpha interferons made by Interferon Sciences, and available from Purdue Frederick Co.); WELLFERON® (interferon alpha, available from Glaxo Wellcome); [alpha]-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha; lymphoblastoid or “natural” interferon; interferon tau; interleukin-2; Interleukin-6; or interleukin-12. Also included are compounds that stimulate the synthesis of interferon in cells including, but not limited to, double stranded RNA, alone or in combination with tobramycin, and Imiquimod.

In a particular embodiment there is provided a method of treating HCV in a human subject comprising the administration of (a) SCY-635, or a pharmaceutically acceptable salt, solvate or hydrate thereof, (b) an alpha interferon, and (c) ribavirin or a pro-drug thereof.

In a further embodiment, provided herein is a method of treating HCV in a human subject comprising the administration of (a) SCY-635, or a pharmaceutically acceptable salt, solvate or hydrate thereof, and (b) an alpha interferon.

In a further embodiment, provided herein is a method of treating HCV in a human subject comprising the administration to the human subject of (a) SCY-635, or a pharmaceutically acceptable salt, solvate or hydrate thereof, (b) an alpha interferon, (c) ribavirin, or a pro-drug thereof and (d) a hepatitis C protease inhibitor. In one aspect of this embodiment the hepatitis C protease inhibitor is an NS3-NS4A inhibitor, for example telaprevir, boceprevir, ITMN-191, MK-7009, PF-00868554, or TMC435350.

In a further embodiment, provided herein is a method of treating HCV in a human subject comprising the administration to the human subject of (a) SCY-635, or a pharmaceutically acceptable salt, solvate or hydrate thereof, (b) an alpha interferon, and (c) a hepatitis C protease inhibitor. In one aspect of this embodiment the hepatitis C protease inhibitor is an NS3-NS4A inhibitor (for example, telaprevir, boceprevir, ITMN-191, MK-7009, PF-00868554, or TMC435350).

In a further embodiment, provided herein is a method of treating HCV in a human subject comprising the administration to the human subject of (a) SCY-635, or a pharmaceutically acceptable salt, solvate or hydrate thereof and (b) an NS3-NS4A hepatitis C protease inhibitor (for example, telaprevir, boceprevir, ITMN-191, MK-7009, PF-00868554, or TMC435350).

In a further embodiment, provided herein is a method of treating HCV in a human subject comprising the administration to the human subject of (a) SCY-635 or a pharmaceutically acceptable salt, solvate or hydrate thereof, (b) an alpha interferon, (c) ribavirin, and (d) a polymerase inhibitor (for example, a nucleoside polymerase inhibitor, such as R-7128).

In a further embodiment, provided herein is a method of treating HCV in a human subject comprising the administration to the human subject of (a) SCY-635 or a pharmaceutically acceptable salt, solvate or hydrate thereof, (b) an alpha interferon, and (c) a polymerase inhibitor (for example, a nucleoside polymerase inhibitor, such as R-7128).

In a further embodiment, provided herein is a method of treating HCV in a human subject comprising the administration to the human subject of (a) SCY-635 or a pharmaceutically acceptable salt, solvate or hydrate thereof, (b) a polymerase inhibitor (for example, a nucleoside polymerase inhibitor, such as R-7128).

In a further embodiment, provided herein is a method of treating HCV in a human subject comprising the administration to the human subject of (a) SCY-635 or a pharmaceutically acceptable salt, solvate or hydrate thereof, (b) an alpha interferon, (c) an NS3-NS4A hepatitis C protease inhibitor (for example telaprevir, boceprevir, ITMN-191, MK-7009, PF-00868554, or TMC435350), and (d) a polymerase inhibitor (for example, a nucleoside polymerase inhibitor, such as R-7128).

In a further embodiment, provided herein is a method of treating HCV in a human subject comprising the administration to the human subject of (a) SCY-635 or a pharmaceutically acceptable salt, solvate or hydrate thereof, (b) an NS3-NS4A hepatitis C protease inhibitor (for example, telaprevir, boceprevir, ITMN-191, MK-7009, PF-00868554, or TMC435350), and (c) a polymerase inhibitor (for example a nucleoside polymerase inhibitor, such as R-7128).

In a further embodiment, provided herein is a method of treating HCV in a human subject comprising the administration to the human subject of (a) SCY-635, or a pharmaceutically acceptable salt, solvate or hydrate thereof, and (b) ribavirin, or a pro-drug thereof.

In a further embodiment, provided herein is a method of treating HCV in a human subject comprising the administration to the human subject of (a) SCY-635, or a pharmaceutically acceptable salt, solvate or hydrate thereof, (b) ribavirin, or a pro-drug thereof, and (c) a hepatitis C protease inhibitor. In one aspect of this embodiment the hepatitis C protease inhibitor is an NS3-NS4A inhibitor, for example telaprevir, boceprevir, ITMN-191, MK-7009, PF-00868554, or TMC435350.

In a further embodiment, provided herein is a method of treating HCV in a human subject comprising the administration to the human subject of (a) SCY-635 or a pharmaceutically acceptable salt, solvate or hydrate thereof, (b) ribavirin, or a pro-drug thereof, and (c) a polymerase inhibitor (for example a nucleoside polymerase inhibitor, such as R-7128).

In a further embodiment, provided herein is a method of treating HCV in a human subject comprising the administration to the human subject of (a) SCY-635, or a pharmaceutically acceptable salt, solvate or hydrate thereof, (b) ribavirin, or a pro-drug thereof, (c) a hepatitis C protease inhibitor, and (d) a polymerase inhibitor (for example a nucleoside polymerase inhibitor, such as R-7128). In one aspect of this embodiment the hepatitis C protease inhibitor is an NS3-NS4A inhibitor, for example telaprevir, boceprevir, ITMN-191, MK-7009, PF-00868554, or TMC435350.

In some aspects, the method includes the administration of agents over two phases, an initial phase and a secondary phase. For instance, the initial phase can be a period of less than about 12 or 24 weeks and the secondary phase can be greater than or equal to about 12 weeks, e.g., the secondary phase can be between about 12-36 weeks. In certain embodiments, the secondary phase is 12 weeks. In still other embodiments, the secondary phase is 36 weeks. In certain embodiments, the sum of the initial and secondary phase is about 24 to 48 weeks (such as 24, 36, or 48 weeks). In some embodiments, the initial and secondary phases can be identical in duration.

SCY-635 may be administered in either the initial phase, the secondary phase, or both phases. In some embodiments, SCY-635 is administered only in the initial phase. When SCY-635 is administered only in the initial phase, SCY-635 may be administered alone or in combination with other agents, and one or more agents may be administered in the secondary phase. The other agents can be one or more anti-viral agents, one or more other agents described herein, or combinations thereof. In some embodiments, the specific agents administered in the initial and secondary phases are identical.

In still other embodiments, the method includes the administration of SCY-635 for four to twenty six weeks in combination with pegylated interferon alpha-2b (Peg-IFN) (initial phase) followed by 22 weeks of administration of a combination of Peg-IFN and ribavirin (secondary phase). In other embodiments, the method includes the administration of SCY-635 for 26 weeks in combination with Peg-IFN (initial phase) followed by 22 weeks of administration of a combination of Peg-IFN and ribavirin (secondary phase). In still other embodiments, the method includes the administration of SCY-635 for 12 weeks in combination with Peg-IFN and ribavirin (initial phase) followed by 36 weeks of administration of a combination of Peg-IFN and ribavirin (secondary phase). In other embodiments, the method includes the administration of SCY-635 for 12 weeks in combination with Peg-IFN and ribavirin (initial phase) followed by 14 weeks of administration of a combination of Peg-IFN and ribavirin (secondary phase).

In further embodiments, the method includes the administration of SCY-635 for four to twenty six weeks in combination with Peg-IFN, ribavirin and a protease inhibitor (initial phase) followed by 22 weeks of administration of a combination of Peg-IFN and ribavirin (secondary phase). In other embodiments, the method includes the administration of SCY-635 and a protease inhibitor for 12 weeks in combination with Peg-IFN (initial phase) followed by 22 weeks of administration of a combination of Peg-IFN and ribavirin (secondary phase). In still other embodiments, the method includes the administration of SCY-635 and a protease inhibitor for 12 weeks in combination with Peg-IFN and ribavirin (initial phase) followed by 36 weeks of administration of a combination of Peg-IFN and ribavirin (secondary phase). In still further embodiments, the method includes the administration of SCY-635 and a protease inhibitor for 12 weeks in combination with Peg-IFN and ribavirin (initial phase) followed by 14 weeks of administration of a combination of Peg-IFN and ribavirin (secondary phase). In still further embodiments, the method includes the administration of SCY-635 and a protease inhibitor for 12 weeks in combination with Peg-IFN and ribavirin (initial phase) followed by 14 weeks of administration of a combination of SCY-635, Peg-IFN and ribavirin (secondary phase). In still further embodiments, the method includes the administration of SCY-635 and a protease inhibitor for 12 weeks in combination with Peg-IFN (initial phase) followed by 14 weeks of administration of a combination of SCY-635 and Peg-IFN (secondary phase).

### Pharmaceutical Compositions and Unit Dosage Formulations

Pharmaceutical compositions and single unit dosage forms comprising SCY-635, or a pharmaceutically acceptable salt, solvate or hydrate thereof, are also provided herein. Individual dosage forms may be suitable for oral, mucosal (including sublingual, buccal, rectal, nasal, or vaginal) or parenteral (including subcutaneous, intramuscular, bolus injection, intraarterial, or intravenous) administration. Preferred pharmaceutical compositions and single unit dosage forms are suitable for oral administration.

In one embodiment, the pharmaceutical composition is a solid oral dosage form. In one embodiment, the pharmaceutical composition is a liquid oral dosage form. In a particular embodiment, provided herein are doses, unit dosage formulations and pharmaceutical compositions wherein SCY-635, or a pharmaceutically acceptable salt, solvate or hydrate thereof, is orally bioavailable. Advantages of oral administration can include ease of administration, higher human subject compliance with the dosing regimen, clinical efficacy, fewer complications, shorter hospital stays, and overall cost savings.

In another embodiment, provided herein are unit dosage formulations that comprise between about 30 mg and about 1400 mg, between about 100 mg and about 1000 mg, between about 200 mg and about 1000 mg, or between about 250 mg and about 1000 mg of SCY-635, or a pharmaceutically acceptable salt, solvate or hydrate thereof. In one embodiment, the unit dosage formulation comprises SCY-635, or a pharmaceutically acceptable salt, solvate or hydrate thereof, and one or more carriers or excipients suitable for suspension in a pharmaceutically acceptable solvent (e.g., water, milk, a carbonated beverage, juice, apple sauce, baby food or baby formula) in a bottle.

In another embodiment, provided herein are unit dosage formulations that comprise about 35 mg, about 50 mg, about 70 mg, about 100 mg, about 125 mg, about 140 mg, about 175 mg, about 200 mg, about 250 mg, about 280 mg, about 350 mg, about 500 mg, about 560 mg, about 700 mg, about 750 mg, about 1000 mg or about 1400 mg of SCY-635, or a pharmaceutically acceptable salt, solvate or hydrate thereof. Preferred unit dosage formulations comprise about 125 mg, about 250, about 300 mg, about 500 mg, or about 1000 mg of SCY-635, or a pharmaceutically acceptable salt, solvate or hydrate thereof. In one embodiment, the unit dosage formulation comprises SCY-635, or a pharmaceutically acceptable salt, solvate or hydrate thereof, and one or more carriers or excipients suitable for suspension in a pharmaceutically acceptable solvent (e.g., water, milk, a carbonated beverage, juice, apple sauce, baby food or baby formula) in a bottle. Preferred unit dosage formulations are capsules, powders and sachets. A particularly preferred unit dosage is a capsule.

Single unit dosage forms suitable for oral administration to a human subject include, but are not limited to: sachets; cachets; tablets; caplets; capsules, such as soft elastic gelatin capsules; troches; lozenges; dispersions; powders; solutions; liquid dosage forms, including suspensions (e.g., aqueous or non-aqueous liquid suspensions); emulsions (e.g., oil-in-water emulsions, or a water-in-oil liquid emulsion); and elixirs. In one embodiment, provided herein is a colloid solution or a solution with additional active agent, above the saturating concentration. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). See also Remington: The Science and Practice of Pharmacy, 21^{st} ed., Lippincott Williams & Wilkins, Philadelphia, Pa. (2005).

In another embodiment, provided herein are anhydrous pharmaceutical compositions and dosage forms comprising SCY-635, or a pharmaceutically acceptable salt, solvate or hydrate thereof. Anhydrous pharmaceutical compositions and dosage forms of the invention can be prepared using anhydrous or low moisture containing ingredients and low moisture or low humidity conditions.

Typical oral dosage forms of the invention are prepared by combining the active ingredient(s) in an intimate admixture with at least one carrier or excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents (e.g., vanilla extract), preservatives, and coloring agents. Examples of excipients suitable for use in solid oral dosage forms (e.g., powders, tablets, sachets, capsules, and caplets) include, but are not limited to, starches, sugars, micro-crystalline cellulose, diluents, granulating agents, lubricants, binders, and disintegrating agents.

In one embodiment, the unit dosage formulations are powder formulations comprising an effective amount of the active agent which are suitable for reconstitution in a pharmaceutically acceptable solvent (e.g., water, milk, a carbonated beverage, juice, apple sauce, baby food or baby formula) and subsequent oral administration. In a particular embodiment, the powder can optionally contain one or more carriers or excipients in combination with the active agent. In another embodiment, the powder can be stored in a sealed container prior to administration or reconstitution. In yet another embodiment, the powder can be encapsulated (e.g., in a gelatin capsule).

The following Examples illustrate the present invention. These examples are not intended, nor are they to be construed, as limiting the scope of the invention. It will be clear that the invention may be practiced otherwise than as particularly described herein. Numerous modifications and variations of the present invention are possible in view of the teachings herein and, therefore, are within the scope of the invention.

### Example 1

The anti-HCV activity of SCY-635 was assessed in a human hepatoma cell line (Huh-7) that contains a Con1 (genotype 1b) bi-cistronic subgenomic replicon. The replicon contains a stable luciferase reporter gene and three cell culture adaptive mutations. The subgenomic replicon contains the 5′ terminus (which includes the HCV Internal Ribosomal Entry Site and the first few amino acids of the HCV core protein) which drives the production of the neomycin phosphotransferase protein. The EMCV IRES element directs the translation of the second cistronic unit that encodes the non-structural proteins NS3, NS4A, NS4B, NS5A, and NS5B. For comparative purposes HCV RNA replication was assessed by quantifying HCV replicon-derived luciferase activity and by quantifying HCV-specific RNA using reverse transcriptase-real time PCR. Duplicate plates were incubated with various concentrations of SCY-635 and were processed either for luciferase activity or for total RNA extraction at 24, 48, 72 and 120 hours after drug treatment. SCY-635 was tested at twelve half-log dilutions ranging from 0.00016 to 50 μM. Stock solutions of each test article were diluted into culture media and added to the plate. After a 72 hour incubation, cells were processed to determine luciferase luminescence as a measurement of antiviral activity. Three replicates were performed and mean values were calculated.

Using linear regression, mean calculated IC₅₀, IC₇₅, IC₉₀ and IC₉₅ values (concentrations required to inhibit 50%, 75%, 90% and 95% of HCV replication) for SCY-635 were 0.091, 0.200, 0.397 and 0.455 μM, respectively. This corresponds to mean effective concentrations, EC₅₀, EC₇₅, EC₉₀ and EC₉₅, for SCY-635 of 121, 264, 501 and 601 ng/ml, respectively.

By inspection of the curves, mean IC₅₀, IC₇₅, IC₉₀, IC₉₅ and IC₉₈ values determined for SCY-635 were 0.081, 0.165, 0.333, 0.410, 0.500 and 1.580 μM, respectively. This corresponds to mean effective concentrations, EC₅₀, EC₇₅, EC₉₀, EC₉₅, EC₉₈ and EC₉₉ for SCY-635, of 107, 217, 441, 542, 661 and 2088 ng/ml, respectively.

Cell cytotoxicity was assessed by measuring the release of lactate dehydrogenase from cell membranes and by measuring total ribosomal RNA. No significant cell cytotoxicity on replicon cells or on the background Huh-7 cell line was observed following 72-hour incubations with SCY-635 at concentrations up to and including 5 μM. Concentrations of SCY-635 that were associated with 50% cell viability, CC₅₀, for replicon cells and for the Huh-7 cell line were 14.2 and 13.7 μM, respectively. Calculation of a selectivity index (CC₅₀/IC₅₀) yielded values of 129.0 and 124.5 for the replicon cells and for the Huh-7 cell line, respectively. SCY-635 was assessed for cytotoxicity in primary human hepatocytes. No significant cytotoxic effects were observed at concentrations up to 20 μM following a 48-hour incubation period. The 50% inhibitory concentration for cell viability of SCY-635 in primary human hepatocytes was 33 μM yielding a selectivity index of approximately 300. SCY-635 was also assessed for cytotoxicity in primary human renal tubular epithelial cells. The 50% inhibitory concentrations for cell viability for cyclosporine A and for SCY-635 against renal tubular epithelial cells were 12.5 μM and 60.6 μM, respectively.

### Example 2

The anti-HCV activity of SCY-635 was also assessed in a human hepatoma cell line that contains a full length genotype 1b derived replicon. Cells were processed following a 72-hour incubation at 37° C. Intracellular RNA from each well was extracted and the level of HCV-specific RNA was determined by reverse transcriptase-real time PCR. Cytotoxic effects were measured by assessing ribosomal RNA as an indication of total cell numbers. Dose-dependent suppression of HCV-specific replication was observed in the absence of significant cell cytotoxicity at concentrations up to 10 μM. EC₅₀ values equaling 0.13, 0.16, and 0.21 μM and corresponding EC₉₀ values equaling 0.57, 0.71, and 0.79 μM were determined in 3 independent experiments. These determinations yielded mean values of 0.17 μM and 0.69 μM for the EC₅₀ and EC₉₀ values, respectively.

### Example 3

For a clinical trial, SCY-635 was formulated as a hard gelatin capsule containing one active ingredient, SCY-635, and four inactive ingredients. The drug product was produced as a dry blend containing SCY-635, pregelatinized starch (Starch 1500), microcrystalline cellulose (Avicel® PH102), colloidal silicon dioxide (Cab-O-Sil® M5P), Magnesium stearate (non-bovine Hyqual®). SCY-635 was also formulated as a concave round tablet containing one active ingredient, SCY-635, and five inactive ingredients. The tablet formulation was produced as a dry blend containing SCY-635, pregelatinized starch (Starch 1500), microcrystalline cellulose (Avicel® PH102), sodium starch glycolate (Explotab), colloidal silicon dioxide (Aerosil® 200), and magnesium stearate.

### Example 4

SCY-635 (given as oral capsules as described in Example 3 above) was examined in a randomized, double-blind, placebo-controlled, multi-dose study to adult volunteers who are chronically infected with hepatitis C (subtype 1). Three cohorts received 100, 200 or 300 milligrams of SCY-635 three times per day (100 mg, 200 mg or 300 mg t.i.d., respectively) per os for 15 consecutive days. The 100 mg t.i.d. cohort was comprised of 7 human subjects (1 placebo and 6 active); the 200 mg t.i.d. cohort was comprised of 6 human subjects (1 placebo and 5 active); and the 300 mg t.i.d. cohort was comprised of 7 human subjects (1 placebo and 6 active).

Human subjects were excluded if they demonstrated evidence of co-infection with HIV-1, HBV, decompensated liver function, hepatocellular carcinoma, ALT values 2.5 times the upper limit of normal, or if they were the recipient of an organ transplant. All human subjects were male (n=20); 75% were African American. Average age was 53.0 years. Average HCV RNA plasma viremia on enrollment was 5,600,000 IU/ml, as measured by the quantitative Roche COBAS taqMan assay. 55% of human subjects were treatment naïve.

Blood and urine samples were collected from human subjects on Days 1, 2, 3, and 14 during the 8-hour interval immediately following administration of the first dose on each specified study day. In addition, blood samples for the measurement of trough drug concentrations were collected from all human subjects immediately prior to the administration of SCY-635 on the mornings of Days 4, 5, 8, 11, 12, and 15, and prior to the evening dose on Day 13.

The study showed that SCY-635 was well tolerated at all dose levels and no serious adverse events were reported during the study. Mild and moderate adverse events were reported; however, no evidence of a dose limiting toxicity was observed.

### Example 5

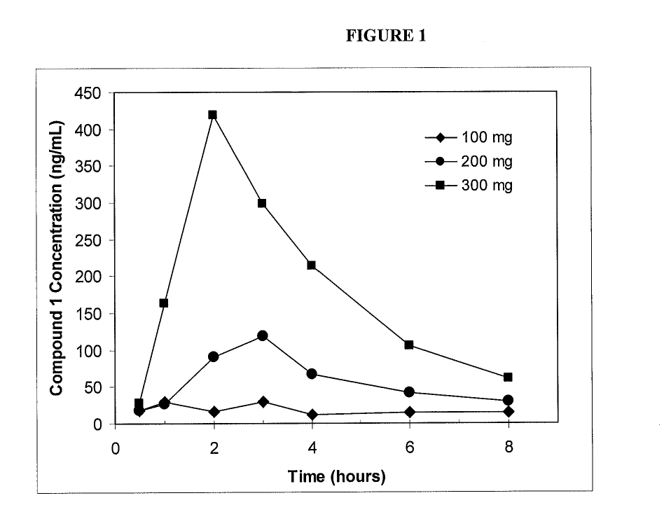

Concentrations of SCY-635 in human blood and plasma samples from the study described in Example 4 above were determined using validated high throughput liquid chromatography/tandem mass spectrometric (HTLC-MS/MS) methods with assay ranges of 20 to 2000 ng/mL for whole blood and 5 to 1000 ng/mL for plasma. FIG. 1 shows the plasma concentrations of SCY-635 (Compound 1) obtained on Day 1 of the Study. These data indicate that doses of SCY-635 above 200 mg t.i.d resulted in super-proportional concentrations of SCY-635 in plasma compared to the 100 mg t.i.d dose.

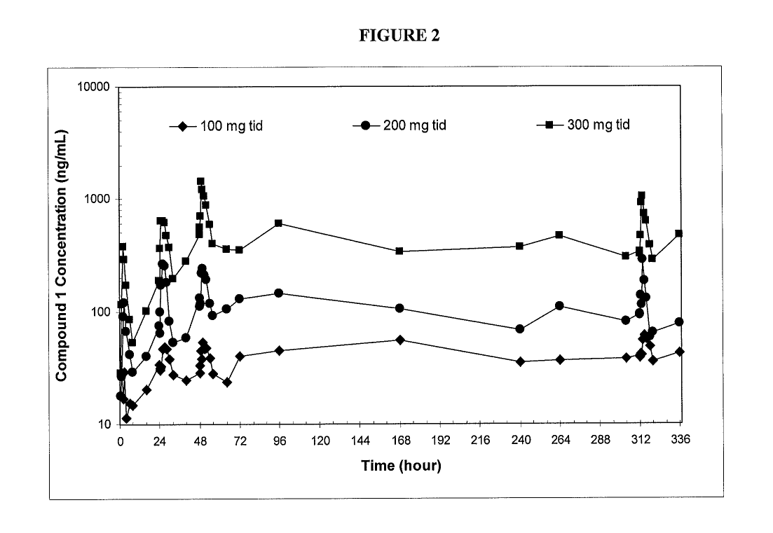

FIG. 2 shows the median plasma concentrations of SCY-635 observed 8 hours after administration of drug and/or immediately prior to the next scheduled dose of SCY-635 for the three cohorts.

### Example 6

Viral load data from the study described in Example 4 above was measured by the quantitative Roche COBAS taqMan assay. The test measures HCV RNA in International Units (IU) per mL using real-time Polymerase Chain Reaction (RT-PCR) technology. It quantitates HCV RNA from 10 to 100,000,000 IU/mL. All clinical viral load samples were assayed at LabCorp.

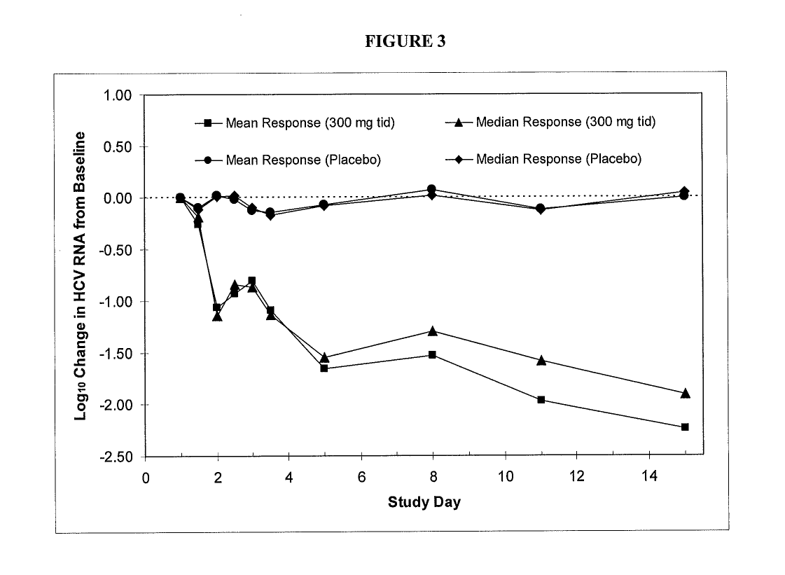

FIG. 3 shows the mean and median viral load response (expressed as a log₁₀ reduction in viral load from baseline) for the 300 mg t.i.d. cohort, in comparison with the human subjects receiving placebo. These results demonstrate that SCY-635 provided a clinically significant reduction in plasma viremia in this study, providing a group mean nadir value of 2.33 log₁₀ below baseline and a group median nadir value of 1.90 log₁₀ below baseline, with a range of nadir values of from 0.8 to 5.5 log₁₀ below baseline. The mean and median nadir values for the 100 mg t.i.d. and 200 mg t.i.d. cohorts were each less than 0.5 log₁₀ below baseline.

### Example 7

SCY-635 (given as oral capsules [400 milligram dose] or tablets [500 and 600 milligram doses] as described in Example 3 above) was examined in a randomized, double-blind, placebo-controlled, pharmacokinetic study in normal healthy adult volunteers. Three cohorts received a single dose of either 400, 500 or 600 milligrams of SCY-635. Each cohort was comprised of 8 human subjects (2 placebo and 6 active). Blood samples were collected from human subjects during the 72-hour interval immediately following administration of a single dose of SCY-635. The study showed that SCY-635 was well tolerated at all dose levels and no serious adverse events were reported during the study. Mild and moderate adverse events were reported; however, no evidence of a dose limiting toxicity was observed.

Concentrations of SCY-635 in human blood and plasma samples from the study described in this Example were determined using validated high pressure liquid chromatography/tandem mass spectrometric (HPLC-MS/MS) methods with assay ranges of 50 to 5000 ng/mL for whole blood and 15 to 2000 ng/mL for plasma. The table below summarizes the results obtained (“BLOQ” means below the limit of quantitation).

| Time | Mean SCY-635 Plasma Concentration (ng/mL) | | |
|---|---|---|---|
| (hours) | 400 mg | 500 mg | 600 mg |
| 0 | BLOQ | BLOQ | BLOQ |
| 0.25 | BLOQ | BLOQ | 51.4 |
| 0.5 | 42.8 | 51.1 | 91.3 |
| 1 | 89.2 | 374 | 241 |
| 2 | 411 | 1190 | 1152 |
| 3 | 788 | 900 | 1184 |
| 4 | 455 | 625 | 790 |
| 6 | 251 | 320 | 461 |
| 8 | 107 | 161 | 249 |
| 12 | 46.3 | 72.0 | 105 |
| 24 | 23.0 | 34.1 | 42.7 |
| 36 | 18.7 | 20.6 | 24.0 |
| 48 | BLOQ | 20.7 | 21.2 |
| 72 | BLOQ | 15.7 | 16.7 |

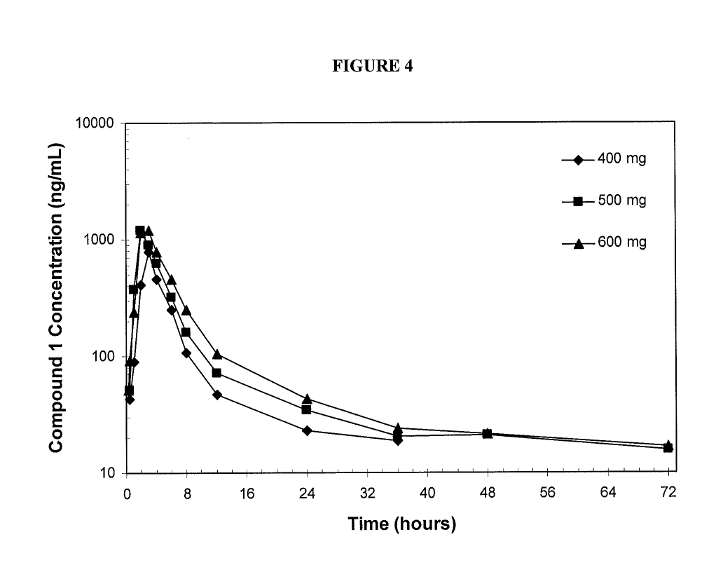

FIG. 4 shows a graphical representation of the measured plasma concentrations of SCY-635 (Compound 1) shown in the table above. These data indicate that all dose levels of SCY-635 were well absorbed.

### Example 8

SCY-635 (given as oral tablets as described in Example 3 above) was examined in a double-blind, placebo-controlled, pharmacokinetic study in normal healthy adult volunteers. A single cohort of two subjects received 1000 milligrams of SCY-635 daily given as 500 milligrams two times per day (500 mg b.i.d.) per os for 14 consecutive days. Blood samples were collected from the subjects on Days 1 and 7 during the 12-hour interval immediately following administration of the first dose on each specified study day. On Day 14, only the morning dose of drug was given and blood samples were collected during the 72-hour interval immediately following administration of the final dose. In addition, blood samples for the measurement of trough drug concentrations were collected from the subjects immediately prior to the administration of SCY-635 on the mornings of Days 2-6 and 8-13. Data collected from the two subjects showed that SCY-635 was well tolerated and no serious adverse events were reported during the study. Mild and moderate adverse events were reported; however, no evidence of a dose limiting toxicity was observed.

Concentrations of SCY-635 in human blood and plasma samples from the two subjects were determined using validated high pressure liquid chromatography/tandem mass spectrometric (HPLC-MS/MS) methods with assay ranges of 50 to 5000 ng/mL for whole blood and 15 to 2000 ng/mL for plasma. The table below shows the mean plasma concentrations of SCY-635 measured over time, where time is expressed relative to the administration of the first dose of study drug on Day 1.

| | Mean SCY-635 |
|---|---|
| Time post first | Plasma Concentration |
| dose (hours) | (ng/mL) |
| 0 | BLOQ |
| 0.25 | BLOQ |
| 0.5 | BLOQ |
| 1 | 87.2 |
| 2 | 1208 |
| 3 | 1005 |
| 4 | 702 |
| 6 | 227 |
| 8 | 114 |
| 12 | 55.8 |
| 24 | 197 |
| 48 | 282 |
| 72 | 435 |
| 96 | 435 |
| 120 | 349 |
| 144 | 306 |
| 144.25 | 310 |
| 144.5 | 471 |
| 145 | 688 |
| 146 | 1745 |
| 147 | 1420 |
| 148 | 1205 |
| 150 | 720 |
| 152 | 546 |
| 156 | 289 |
| 168 | 347 |
| 192 | 446 |
| 216 | 434 |
| 240 | 319 |
| 264 | 411 |
| 288 | 337 |
| 312 | 309 |
| 312.25 | 316 |
| 312.5 | 597 |
| 313 | 1149 |
| 314 | 1780 |
| 315 | 1430 |
| 316 | 1195 |
| 318 | 747 |
| 320 | 482 |
| 324 | 300 |

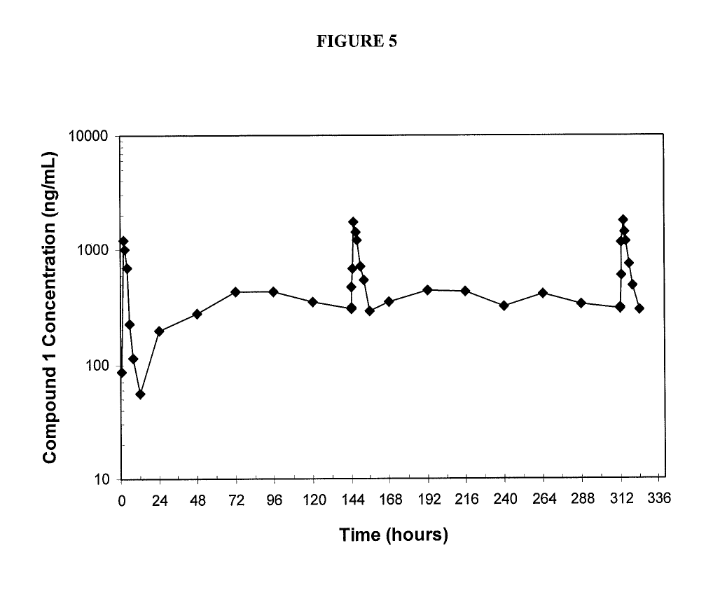

FIG. 5 is a graphical representation of the data in the above table showing the mean plasma concentrations of SCY-635 (Compound 1) over time. These data indicate that SCY-635 administered as 500 milligrams twice daily resulted in trough concentrations (i.e., concentrations obtained in samples collected prior to the morning dose) that exceed an average of 370 ng/mL at steady state.

All publications, patents and patent applications cited in this specification are herein incorporated by reference as if each individual publication, patent or patent application were specifically and individually indicated to be incorporated by reference. While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A method for treating, preventing or managing hepatitis C virus infection or human immunodeficiency virus infection in a human subject infected with, or at risk for infection with, hepatitis C virus or human immunodeficiency virus, the method comprising administering to the human subject the compound 3-[(R)-2-(N,N-dimethylamino)ethylthio-Sar]-4-(gammahydroxymethylleucine)cyclosporine, or a pharmaceutically acceptable salt, solvate or hydrate thereof, in an amount of greater than about 600 mg per day, as a divided dose.

2. A method for treating, preventing or managing hepatitis C virus infection in a human subject infected with, or at risk for infection with, hepatitis C virus, the method comprising administering to the human subject the compound 3-[(R)-2-(N,N-dimethylamino)ethylthio-Sar]-4-(gammahydroxymethylleucine)cyclosporine, or a pharmaceutically acceptable salt, solvate or hydrate thereof, in an amount of greater than about 600 mg per day, as a divided daily dose.

3. The method of claim 2, wherein the compound is administered in an amount of from about 800 mg to about 1600 mg per day.

4. The method of claim 2, wherein the compound is administered in an amount of from about 800 mg to about 1000 mg per day.

5. The method of claim 2, wherein the compound is administered in an amount of about 900 mg per day.

6. The method of claim 2, wherein the compound is administered twice a day.

7. The method of claim 2, wherein the compound is administered three times a day.

8. The method of claim 2, wherein the human subject is co-infected with HIV.

9. The method of claim 2, wherein the compound is administered orally.

10. The method of claim 2, wherein the human subject is infected with HCV genotype 1.

11. The method of claim 2, further comprising administering to the human subject one or more further anti-viral agents.

12. A method for treating, preventing or managing hepatitis C virus infection in a human subject infected with, or at risk for infection with, hepatitis C virus, the method comprising administering to the human subject the compound 3-[(R)-2-(N,N-dimethylamino)ethylthio-Sar]-4-(gammahydroxymethylleucine)cyclosporine, or a pharmaceutically acceptable salt, solvate or hydrate thereof, wherein the compound is administered in amount sufficient to maintain a trough plasma concentration of the compound of greater than about 115 ng/ml at steady state.

13. A method for treating, preventing or managing hepatitis C virus infection in a human subject infected with, or at risk for infection with, hepatitis C virus, the method comprising administering to the human subject the compound 3-[(R)-2-(N,N-dimethylamino)ethylthio-Sar]-4-(gammahydroxymethylleucine)cyclosporine, or a pharmaceutically acceptable salt, solvate or hydrate thereof, wherein the compound is administered in amount sufficient to maintain an average plasma concentration of the compound of greater than about 250 ng/ml at steady state.

14. A method for treating, preventing or managing hepatitis C virus infection in a human subject infected with, or at risk for infection with, hepatitis C virus, the method comprising administering to the human subject the compound 3-[(R)-2-(N,N-dimethylamino)ethylthio-Sar]-4-(gammahydroxymethylleucine)cyclosporine, or a pharmaceutically acceptable salt, solvate or hydrate thereof, in an amount of from about 10 mg/kg to about 50 mg/kg per day, in a divided dose.
